(19) United States
(12) Patent Application Publication
(10) Pub. No.: US 2003/0220237 A1
(43) Pub. Date: Nov. 27, 2003

(54) Pharmaceutical composition

(75) Inventors: Chika Ohnaka, Osaka (JP); Mayumi Nakai, Osaka (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Fujisawa Pharmaceutical Co. Ltd., Osaka-shi (JP)

(21) Appl. No.: 10/408,558

(22) Filed: Apr. 8, 2003

(30) Foreign Application Priority Data
Apr. 22, 2002 (AU) PS1883

(51) Int. Cl. A61K 38/12; A61K 31/724

(52) U.S. Cl. 514/9; 514/58

(57) ABSTRACT

A pharmaceutical composition comprising:

a cyclic peptide compound represented by the following general formula (I):

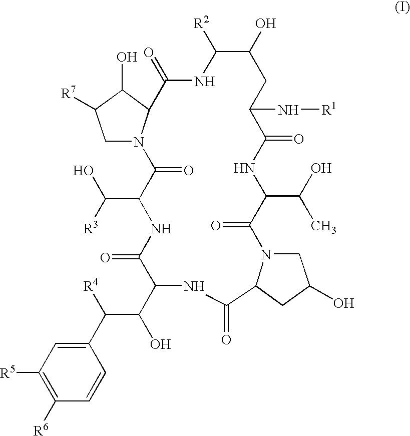

wherein

R¹ is hydrogen or acyl group,

R² is hydrogen, hydroxy or —NH(CH₂)₂NH₂,

R³ is methyl, —CH₂CONH₂, —(CH₂)₂NH₂ or

lower alkylamino(lower) alkyl substituted with one more hydroxy,

R⁴ is hydrogen or hydroxy,

R⁵ is hydrogen, hydroxy, lower alkoxy or hydroxysulfonyloxy,

R⁶ is hydroxy or acyloxy and,

R⁷ is hydrogen or methyl,

or a salt thereof as an active ingredient, and cyclodextrins.

### TECHNICAL FIELD

The present invention relates to a pharmaceutical composition containing a cyclic peptide compound. More particularly, the present invention relates to a pharmaceutical composition containing a cyclic peptide compound or a salt thereof and cyclodextrins.

### BACKGROUND ART

The cyclic peptide compound (I) explained below is disclosed in WO 01/60846, U.S. Pat. No. 5,378,804 and EP 561639.

The cyclic peptide compound (I) or a salt thereof has an antimicrobial activity, particularly an antifungal activity and a β-1,3-glucan synthase inhibiting action, and is useful for preventing and treating various kinds of infectious diseases including Pneumocystis carinii infection, e.g., carinii pneumonia.

There is desired development of pharmaceutical preparations in which the cyclic peptide compound (I) or a salt thereof is comprised.

### DISCLOSURE OF INVENTION

The present invention provides a pharmaceutical composition comprising a cyclic peptide compound (I) or a salt thereof and cyclodextrins.

The cyclic peptide compound of the present invention is represented by the following general formula (I):

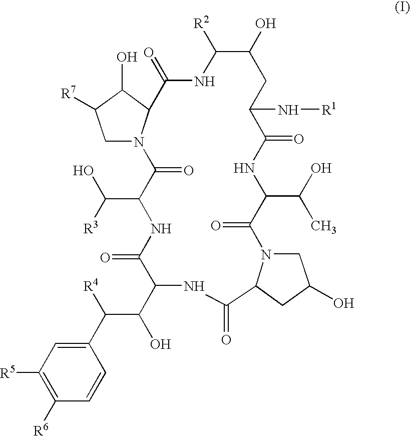

wherein

R¹ is hydrogen or acyl group,

R² is hydrogen, hydroxy or —NH (CH₂)₂NH₂,

R³ is methyl, —CH₂CONH₂, —(CH₂)₂NH₂ or

lower alkylamino(lower)alkyl substituted with one or more hydroxy,

R⁴ is hydrogen or hydroxy,

R⁵ is hydrogen, hydroxy, lower alkoxy or hydroxysulfonyloxy,

R⁶ is hydroxy or acyloxy and,

R⁷ is hydrogen or methyl,

or a salt thereof.

Suitable examples and illustration of the various definitions in the above and subsequent descriptions of the present specification, which the present invention intends to include within the scope thereof, are explained in detail as follows:

The term “lower” is used to intend a group having 1 to 6 carbon atom(s), unless otherwise provided.

Suitable example of “one or more” may be the number of 1 to 6, in which the preferred one may be the number of 1 to 3, and the most preferred one may be the number of 1 or 2.

Suitable example of “halogen” may be fluorine, chlorine, bromine, iodine and the like.

Suitable example of “lower alkoxy” may include straight or branched one such as methoxy, ethoxy, propoxy, isopropoxy, butoxy, isobutoxy, tert-butoxy, pentyloxy, tert-pentyloxy, neo-pentyloxy, hexyloxy, isohexyloxy and the like.

Suitable example of “higher alkoxy” may include straight or branched one such as heptyloxy, octyloxy, 3,5-dimethyloctyloxy, 3,7-dimethyloctyloxy, nonyloxy, decyloxy, undecyloxy, dodecyloxy, tridecyloxy, tetradecyloxy, hexadecyloxy, heptadecyloxy, octadecyloxy, nonadecyloxy, icosyloxy, and the like.

Suitable example of “lower alkyl” may include straight or branched one having 1 to 6 carbon atom(s), such as methyl, ethyl, propyl, isopropyl, butyl, isobutyl, sec-butyl, tert-butyl, pentyl, tert-pentyl, neo-pentyl, hexyl, isohexyl and the like.

Suitable example of “higher alkyl” may include straight or branched one such as heptyl, octyl, 3,5-dimethyloctyl, 3,7-dimethyloctyl, nonyl, decyl, undecyl, dodecyl, tridecyl, tetradecyl, hexadecyl, heptadecyl, octadecyl, nonadecyl, icosyl, and the like.

Suitable example of “aryl” and “ar” moiety may include phenyl which may have lower alkyl (e.g., phenyl, mesityl, xylyl, tolyl, etc.), naphthyl, anthryl, indanyl, fluorenyl, and the like, and this “aryl” and “ar” moiety may have one or more halogen.

Suitable example of “aroyl” may include benzoyl, toluoyl, naphthoyl, anthrylcarbonyl, and the like.

Suitable example of “heterocyclic group” may include

unsaturated 3 to 8-membered (more preferably 5 or 6-membered) heteromonocyclic group containing 1 to 4 nitrogen atom(s), for example, pyrrolyl, pyrrolinyl, imidazolyl, pyrazolyl, pyridyl, dihydropyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.), tetrazolyl (e.g. 1H-tetrazolyl, 2H-tetrazolyl, etc.), etc.;

saturated 3 to 8-membered (more preferably 5 or 6-membered) heteromonocyclic group containing 1 to 4 nitrogen atom(s), for example, pyrrolidinyl, imidazolidinyl, piperidyl, piperazinyl, azetidinyl, etc.;

unsaturated condensed heterocyclic group containing 1 to 4 nitrogen atom(s), for example, indolyl, isoindolyl, indolinyl, indolizinyl, benzimidazolyl, quinolyl, isoquinolyl, indazolyl, benzotriazolyl, etc.;

unsaturated 3 to 8-membered (more preferably 5 or 6-membered) heteromonocyclic group containing 1 or 2 oxygen atom(s) and 1 to 3nitrogenatom(s) , for example, oxazolyl, isoxazolyl, oxadiazolyl (e.g., 1,2,4-oxadiazolyl, 1,3,4-oxadiazolyl, 1,2,5-oxadiazolyl, etc.), etc.;

saturated 3 to 8-membered (more preferably 5 or 6-membered) heteromonocyclic group containing 1 or 2 oxygen atom(s) and 1 to 3 nitrogen atom(s) , for example, morpholinyl, sydnonyl, morpholino, etc.;

unsaturated condensed heterocyclic group containing 1 or 2 oxygen atom(s) and 1 to 3nitrogen atom(s), for example, benzoxazolyl, benzoxadiazolyl, etc.;

unsaturated 3 to 8-membered (more preferably 5 or 6- membered) heteromonocyclic group containing 1 or 2 sulfur atom(s) and 1 to 3 nitrogen atom(s), for example, thiazolyl, isothiazolyl, thiadiazolyl (e.g., 1,2,3-thiadiazolyl, 1,2,4-thiadiazolyl, 1,3,4-thiadiazolyl, 1,2,5-thiadiazolyl, etc.), dihydrothiazinyl, etc.;

saturated 3 to 8-membered (more preferably 5 or 6-membered) heteromonocyclic group containing 1 or 2 sulfur atom(s) and 1 to 3 nitrogen atom(s), for example thiazolidinyl, thiomorpholinyl, thiomorpholino, etc.;

unsaturated 3 to 8-membered (more preferably 5 or 6-membered) heteromonocyclic group containing 1 or 2 sulfur atom(s), for example, thienyl, dihydrodithiinyl, dihydrodithionyl, etc.;

unsaturated condensed heterocyclic group containing 1 or 2 sulfur atom(s) and 1 to 3 nitrogen atom(s), for example, benzothiazolyl, benzothiadiazolyl, imidazothiadiazolyl, etc.;

unsaturated 3 to 8-membered (more preferably 5 or 6-membered) heteromonocyclic group containing an oxygen atom, for example, furyl etc.;

saturated 3 to 8-membered (more preferably 5 or 6-membered) heteromonocyclic group containing 1 or 2 oxygen atom(s), for example, tetrahydrofuran, tetrahydropyran, dioxacyclopentane, dioxacyclohexane, etc.;

unsaturated 3 to 8-membered (more preferably 5 or 6-membered) heteromonocyclic group containing an oxygen atom and 1 or 2 sulfur atom(s), for example, dihydrooxathiinyl, etc.;

unsaturated condensed heterocyclic group containing 1 or 2 sulfur atom(s), for example benzothienyl, benzodithiinyl, etc.;

unsaturated condensed heterocyclic group containing an oxygen atom and 1 or 2 sulfur atom(s), for example, benzoxathiinyl, etc.; and the like, and this “heterocyclic group” may have one or more suitable substituent(s) selected from the group consisting of lower alkyl, oxo, cyclo(lower)alkyl, hydroxy(lower)alkyl, carboxy(lower)alkanoyl which may have amino and heterocycliccarbonyl.

Suitable example of “cyclo(lower)alkyl” may include cyclopropyl, cyclobutyl, cyclopentyl, cyclohexyl, and the like, and this “cyclo(lower)alkyl” may have one or more lower alkyl.

Suitable example of “cyclo(lower)alkyloxy” may include cyclopropyloxy, cyclobutyloxy, cyclopentyloxy, cyclohexyloxy, and the like.

Suitable example of “acyl group” may include carboxy; carbamoyl; mono or di (lower) alkylcarbamoyl (e. g., methylcarbamoyl, dimethylcarbamoyl, ethylcarbamoyl, diethylcarbamoyl, etc.); aliphatic acyl, aromatic acyl, arylaliphatic acyl and heterocyclic-aliphaticacyl derived from carboxylic acid, carbonic acid, carbamic acid, sulfonic acid; and the like.

Aliphatic acyl such as lower or higher alkanoyl (e.g., formyl, acetyl, propanoyl, butanoyl, 2-methylpropanoyl, pentanoyl, 2,2-dimethylpropanoyl, hexanoyl, heptanoyl, octanoyl, nonanoyl, decanoyl, undecanoyl, dodecanoyl, tridecanoyl, tetradecanoyl, pentadecanoyl, hexadecanoyl, heptadecanoyl, octadecanoyl, nonadecanoyl, icosanoyl, etc.); lower or higher alkoxycarbonyl (e.g., methoxycarbonyl, ethoxycarbonyl, t-butoxycarbonyl, t-pentyloxycarbonyl, heptyloxycarbonyl, etc.); lower alkenyloxycarbonyl (e.g., vinyloxycarbonyl, propenyloxycarbonyl, allyloxycarbonyl, butenyloxycarbonyl, butedienyloxycarbonyl, pentenyloxycarbonyl, hexenyloxycarbonyl, etc.); lower or higher alkylsulfonyl (e.g., methylsulfonyl, ethylsulfonyl, etc.); lower or higher alkoxysulfonyl (e.g., methoxysulfonyl, ethoxysulfonyl, etc.); or the like;

Aromatic acyl such as aroyl (e.g., benzoyl, toluoyl, naphthoyl, etc.) ;ar(lower)alkanoyl [e.g., phenyl(C₁-C₆)alkanoyl (e.g., phenylacetyl, phenylpropanoyl, phenylbutanoyl, phenylisobutanoyl, phenylpentanoyl, phenylhexanoyl, etc.), naphthyl (C₁-C₆) alkanoyl (e.g., naphthylacetyl, naphthylpropanoyl, naphthylbutanoyl, etc.), etc.], ar(lower)alkenoyl [e.g., phenyl(C₃-C₆)alkenoyl (e g., phenylpropenoyl, phenylbutenoyl, phenylmethacryloyl, phenylpentanoyl, phenylhexenoyl, etc.), naphthyl(C₃-C₆)alkenoyl (e.g., naphthylpropenoyl, naphthylbutenoyl, etc.), etc.]; ar(lower)alkoxycarbonyl [e.g., phenyl(C₁-C₆)alkoxycarbonyl (e.g., benzyloxycarbonyl, etc.), fluorenyl(C₁-C₆)alkoxy-carbonyl (e.g., fluorenylmethyloxycarbonyl, etc.), etc.]; aryloxycarbonyl (e.g., phenoxycarbonyl, naphthyloxycarbonyl, etc.); aryloxy(lower)alkanoyl (e.g., phenoxyacetyl, phenoxypropionyl, etc.); arylcarbamoyl (e.g., phenylcarbamoyl, etc.); arylthiocarbamoyl (e.g., phenylthiocarbamoyl, etc.); arylglyoxyloyl (e.g., phenylglyoxyloyl, naphthylglyoxyloyl, etc.); arylsulfonyl which may have 1 to 4 lower alkyl (e.g.,phenylsulfonyl, p-tolylsulfonyl, etc.); aroyl (e.g., benzoyl) substituted with one or more suitable substituent(s); or the like;

Heterocyclic acyl such as heterocycliccarbonyl; heterocyclic(lower)alkanoyl (e.g., heterocyclicacetyl, heterocyclicpropanoyl, heterocyclicbutanoyl, heterocyclicpentanoyl, heterocyclichexanoyl, etc.); heterocyclic(lower)alkenoyl (e.g., heterocyclicpropenoyl, heterocyclicbutenoyl, heterocyclicpentenoyl, heterocyclichexenoyl, etc.); heterocyclicglyoxyloyl; or the like; in which suitable “heterocyclic” moiety in the terms “heterocycliccarbonyl”, “heterocyclic(lower)alkanoyl”, “heterocyclic(lower)alkenoyl” and “heterocyclicglyoxyloyl” can be referred to aforementioned “heterocyclic” moiety.

Suitable example of “acyl group” of R¹ can be referred to aforementioned “acyl group”, in which the preferred one may be lower alkoxycarbonyl, higher alkanoyl and benzoyl substituted with one or more suitable substituent(s).

Suitable example of “suitable substituent(s)” in the term of “benzoyl substituted with one or more suitable substituent (s)” may be thiadiazolyl substituted with phenyl having phenyl substituted with morphlino having lower alkyl,

thiadiazolyl substituted with phenyl having a suitable substituent selected from the group consisting of lower alkoxy(lower)alkoxy and lower alkoxy(higher)alkoxy,

piperazinyl substituted with phenyl having piperidyl substituted with a suitable substituent selected from the group consisting of phenyl having lower alkoxy(lower)alkoxy, cyclo(lower)alkyloxy and lower alkoxy(lower)alkylthio,

piperazinyl substituted with phenyl having phenyl substituted with morpholino having lower alkyl,

imidazothiadiazolyl substituted with phenyl having piperidyl substituted with a suitable substituent selected from the group consisting of lower alkoxy(lower)alkoxy and lower alkoxy(lower)alkylthio,

imidazothiadiazolyl substituted with phenyl having lower alkoxy(lower)alkoxy,

phenyl subsutituted with piperazinyl having phenyl substituted with morpholino having lower alkyl,

isoxazolyl substituted with phenyl having lower alkoxy(lower)alkoxy,

isoxazolyl substituted with phenyl having higher alkoxy substituted with morpholino having lower alkyl,

thiadiazolyl substituted with phenyl having piperazinyl substituted with cyclo (lower) alkyl which has one or more suitable substituent(s) selected from the group consisting of lower alkyl, lower alkenyl, lower alkoxy(higher)alkoxy and phenyl,

thiadiazolyl substituted with phenyl having piperazinyl substituted with lower alkyl having cyclo(lower)alkyl,

thiadiazolyl substituted with phenyl having piperidyl substituted with one or more suitable substituent (s) selected from the group consisting of cyclo(lower)alkyloxy, lower alkoxy(lower)alkoxy and lower alkoxy(lower)alkoxy(lower)alkyl,

thiadiazolyl substituted with phenyl having piperidyl substituted with cyclo(lower)alkyl and lower alkoxy,

thiadiazolyl substituted with pyridyl having piperazinyl substituted with cyclo(lower)alkyl having lower alkyl,

imidazothiadiazolyl substituted with phenyl having piperidyl substituted with cyclo(lower)alkyl,

imidazothiadiazolyl substituted with phenyl having piperazinyl substituted with cyclo(lower)alkyl having lower alkyl or,

phenyl substituted with piperazinyl having cyclo(lower)alkyl substituted with one or more suitable substituent(s) selected from the group consisting of cyclo(lower)alkyl which may have lower alkoxy, lower alkyl, lower alkoxy and phenyl which may have lower alkoxy,

in which the preferred one may be thiadiazolyl substituted with phenyl having phenyl substituted with morpholino having dimethyl,

thiadiazolyl substituted with phenyl having a substituent selected from the group consisting of methoxyhexyloxy and methoxyheptyloxy,

piperazinyl substituted with phenyl having piperidyl substituted with a substituent selected from the group consisting of phenyl having methoxybutoxy, cyclohexyloxy and methoxyhexylthio,

piperazinyl substituted with phenyl having phenyl substituted with morpholino having dimethyl,

imidazothiadiazolyl substituted with phenyl having piperidyl substituted with a substituent selected from the group consisting of methoxypropoxy, methoxybutoxy, methoxypentyloxy and methoxyhexylthio,

imidazothiadiazolyl substituted with phenyl having methoxybutoxy,

phenyl subsutituted with piperazinyl having phenyl substituted with morpholino having dimethyl,

isoxazolyl substituted with phenyl having methoxyhexyloxy,

isoxazolyl substituted with phenyl having heptyloxy substituted with morphlino having dimethyl,

thiadiazolyl substituted with phenyl having piperazinyl substituted with cyclohexyl which has one or two substituent(s) selected from the group consisting of methyl, methylene, methoxyheptyloxy, methoxyoctyloxy and phenyl,

thiadiazolyl substituted with phenyl having piperazinyl substituted with methyl which has a substituent selected from the group consisting of cyclopentyl and cyclohexyl,

thiadiazolyl substituted with phenyl having piperidyl substituted with one or two substituent (s) selected from the group consisting of cyclohexyl, methoxy, cyclohexyloxy, methoxypentyloxy, methoxyhexyloxy, methoxybutoxymethyl and methoxypentyloxymethyl,

thiadiazolyl substituted with pyridyl having piperazinyl substituted with cyclohexyl which has a substituent selected from the group consisting of methyl and ethyl,

imidazothiadiazolyl substituted with phenyl having piperidyl substituted with cyclohexyl,

imidazothiadiazolyl substituted with phenyl having piperazinyl substituted with cyclohexyl having methyl,

phenyl substituted with piperazinyl having cyclohexyl substituted with one or two substituent (s) selected from the group consisting of ethyl, t-butyl, methoxy, cyclopentyl, cyclohexyl which may have methoxy or dimethyl, and phenyl which may have methoxy or,

phenyl substituted with phenyl having lower alkoxy.

The suitable example of “acyl group” for R¹ may be

benzoyl which has thiadiazolyl substituted with phenyl having phenyl substituted with morpholino having dimethyl,

benzoyl which has thiadiazolyl substituted with phenyl having a substituent selected from the group consisting of methoxyhexyloxy and methoxyheptyloxy,

benzoyl which has piperazinyl substituted with phenyl having piperidyl substituted with a substituent selected from the group consisting of phenyl having methoxybutoxy, cyclohexyloxy and methoxyhexylthio,

benzoyl which has piperazinyl substituted with phenyl having phenyl substituted with morpholino having dimethyl,

benzoyl which has imidazothiadiazolyl substituted with phenyl having piperidyl substituted with a substituent selected from the group consisting of methoxypropoxy, methoxybutoxy, methoxypentyloxy and methoxyhexylthio,

benzoyl which has imidazothiadiazolyl substituted with phenyl having methoxybutoxy,

benzoyl which has phenyl subsutituted with piperazinyl having phenyl substituted with morpholino having dimethyl,

benzoyl which has isoxazolyl substituted with phenyl having methoxyhexyloxy,

benzoyl which has isoxazolyl substituted with phenyl having heptyloxy substituted with morphlino having dimethyl,

benzoyl which has thiadiazolyl substituted with phenyl having piperazinyl substituted with cyclohexyl which has one or two substituent(s) selected from the group consisting of methyl, methylene, methoxyheptyloxy, methoxyoctyloxy and phenyl,

benzoyl which has thiadiazolyl substituted with phenyl having piperazinyl substituted with methyl which has a substituent selected from the group consisting of cyclopentyl and cyclohexyl,

benzoyl which has thiadiazolyl substituted with phenyl having piperidyl substituted with one or two substituent(s) selected from the group consisting of cyclohexyl, methoxy, cyclohexyloxy, methoxypentyloxy, methoxybutoxymethyl and methoxypentyloxymethyl,

benzoyl which has thiadiazolyl substituted with pyridyl having piperazinyl substituted with cyclohexyl which has a substituent selected from the group consisting of methyl and ethyl,

benzoyl which has imidazothiadiazolyl substituted with phenyl having piperidyl substituted with a substituent selected from the group consisting of methoxyhexyloxy, cyclohexyl and methoxyhexyl,

benzoyl which has imidazothiadiazolyl substituted with phenyl having piperazinyl substituted with cyclohexyl having methyl,

benzoyl which has phenyl substituted with piperazinyl having cyclohexyl substituted with one or two substituent(s) selected from the group consisting of ethyl, t-butyl, methoxy, cyclopentyl, cyclohexyl which may have methoxy or dimethyl, and phenyl which may have methoxy or,

benzoyl which has phenyl substituted with phenyl having pentyloxy.

The suitable example of “acyloxy” may be lower alkanoyloxy or phosphonooxy, and the more preferred one may be acetyloxy and phosphonooxy.

Particularly, the preferred examples of the cyclic peptide compound (I) of the present invention are as follows:

the compound (I), wherein

R¹ is hydrogen, lower alkoxycarbonyl, higher alkanoyl or benzoyl substituted with one or more suitable substituent(s),

R² is hydrogen or —NH(CH₂)₂NH₂,

R³ is methyl, —(CH₂)₂NH₂ or

lower alkylamino(lower)alkyl substituted with one more hydroxy,

R⁴ is hydrogen or hydroxy,

R⁵ is hydrogen, hydroxy or hydroxysulfonyloxy,

R⁶ is hydroxy or phosphonooxy and,

R⁷ is hydrogen or methyl.

And, more preferred one may be the compound (I) wherein

R¹ is benzoyl substituted with a suitable substituent selected from the group consisting of

thiadiazolyl substituted with phenyl having phenyl substituted with morphlino having lower alkyl,

thiadiazolyl substituted with phenyl having a suitable substituent selected from the group consisting of lower alkoxy(lower)alkoxy and lower alkoxy(higher)alkoxy,

piperazinyl substituted with phenyl having piperidyl substituted with a suitable substituent selected from the group consisting of phenyl having lower alkoxy(lower)alkoxy, cyclo(lower)alkyloxy and lower alkoxy(lower)alkylthio,

piperazinyl substituted with phenyl having phenyl substituted with morpholino having lower alkyl,

imidazothiadiazolyl substituted with phenyl having piperidyl substituted with a suitable substituent selected from the group consisting of lower alkoxy(lower)alkoxy and lower alkoxy(lower)alkylthio,

imidazothiadiazolyl substituted with phenyl having lower alkoxy(lower)alkoxy,

phenyl subsutituted with piperazinyl having phenyl substituted with morpholino having lower alkyl,

isoxazolyl substituted with phenyl having lower alkoxy(lower)alkoxy,

isoxazolyl substituted with phenyl having higher alkoxy substituted with morpholino having lower alkyl,

thiadiazolyl substituted with phenyl having piperazinyl substituted with cyclo (lower) alkyl which has one or more suitable substituent(s) selected from the group consisting of lower alkyl, lower alkenyl, lower alkoxy(higher)alkoxy and phenyl,

thiadiazolyl substituted with phenyl having piperazinyl substituted with lower alkyl having cyclo(lower)alkyl,

thiadiazolyl substituted with phenyl having piperidyl substituted with one or more suitable substituent (s) selected from the group consisting of cyclo(lower)alkyloxy, lower alkoxy(lower)alkoxy and lower alkoxy(lower)alkoxy(lower)alkyl,

thiadiazolyl substituted with phenyl having piperidyl substituted with cyclo(lower)alkyl and lower alkoxy,

thiadiazolyl substituted with pyridyl having piperazinyl substituted with cyclo(lower)alkyl having lower alkyl,

imidazothiadiazolyl substituted with phenyl having piperidyl substituted with cyclo(lower) alkyl,

imidazothiadiazolyl substituted with phenyl having piperazinyl substituted with cyclo(lower) alkyl having lower alkyl,

phenyl substituted with piperazinyl having cyclo(lower)alkyl substituted with one or more suitable substituent(s) selected from the group consisting of cyclo(lower)alkyl which may have lower alkoxy, lower alkyl, lower alkoxy and phenyl which may have lower alkoxy or,

phenyl substituted with phenyl having lower alkoxy.

And, the most preferred one may be the compound (I) Wherein

R¹ is benzoyl which has thiadiazolyl substituted with phenyl having piperazinyl substituted with cyclo(lower)alkyl which has lower alkyl,

benzoyl which has thiadiazolyl substituted with phenyl having piperidyl substituted with cyclo(lower)alkyloxy,

benzoyl which has phenyl substituted with piperazinyl having cyclo(lower)alkyl substituted with cyclo(lower)alkyl and lower alkoxy,

benzoyl which has thiadiazolyl substituted with phenyl having piperidyl substituted with cyclo(lower)alkyl,

R² is hydrogen,

R³ is lower alkylamino(lower)alkyl substituted with one more hydroxy,

R⁴ is hydrogen or hydroxy,

R⁵ is hydroxy or hydroxysulfonyloxy

R⁶ is hydroxy, and

R⁷ is methyl.

The suitable salt of the cyclicpeptide compound (I) is soluble in water, and a pharmaceutically acceptable salt including salt with base and acid addition salt. Such a salt may be prepared by treating the cyclic peptide compound (I) with an appropriate base or acid according to the conventional method.

As a salt with bases, may be mentioned salt with inorganic base such as alkali metal salt (e.g., sodium salt, potassium salt, etc.), alkaline earth metal salt (e.g., calcium salt, magnesium salt, etc.), ammonium salt and the like; salt with organic base such as organic amine salt (e.g., triethylamine salt, diisopropylethylamine salt, pyridine salt, picoline salt, ethanolamine salt, triethanolamine salt, dicyclohexylamine salt, N,N′-dibenzylethylenediamine salt, etc.); and the like.

As acid addition salt, may be mentioned inorganic acid addition salt (e.g., hydrochloride, hydrobromide, sulfate, phosphate, etc.) ; and organic carboxylic or sulfonic acid addition salt (e.g., formate, acetate, trifluoroacetate, maleate, tartrate, fumarate, methanesulfonate, benzenesulfonate, toluenesulfonate, etc.). Further, may also be mentioned salt with basic or acidic amino acid (e.g., salt with arginine, aspartic acid, glutamic acid, etc.).

The cyclic peptide compound (I) of the present invention may also include possible conformers and a pair or more of stereoisomers such as geometric isomers and optical isomers which may exist due to asymmetric carbon atoms.

The amount of the cyclic peptide compound (I) orasalt thereof contained in the composition for a single unit dosage of the present invention is 0.1 to 400 mg, more preferably 1 to 200 mg, still more preferably 5 to 100 mg, specifically 5, 10, 15, 20, 25, 30, 35, 40, 45, 50, 55, 60, 65, 70, 75, 80, 85, 90, 95 and 100 mg.

The preferred composition of the present invention comprises the cyclic peptide compound (I) ora salt thereof and cyclodextrins, optionally adding a pH adjuster and/or a pH buffer.

The suitable examples of the cyclodextrins are α-cyclodextrins, β-cyclodextrins(e.g., β-cyclodextrin, carboxymethyl-β-cyclodextrin, carboxymethyl-ethyl-β-cyclodextrin, diethyl-β-cyclodextrin, dimethyl-β-cyclodextrin, methyl-β-cyclodextrin, random methyl-β-cyclodextrin, glucosyl-β-cyclodextrin, maltosyl-β-cyclodextrin, hydroxyethyl-β-cyclodextrin, hydroxypropyl-β-cyclodextrin, sulfobutylether-β-cyclodextrin, etc.), γ-cyclodextrins or δ-cyclodextrins, or a combination thereof.

The amount of the cyclodextrins used in the pharmaceutical composition of the present invention should be at least sufficient for stabilizing the cyclic peptide compound (I) or a salt thereof in the composition. In order to stabilize the cyclic peptide compound (I), one part by weight of the cyclodextrins with respect to one part by weight of the cyclic peptide compound (I) or a salt thereof in the present composition is sufficient at least. The cyclodextrins may also serve as a carrier or an excipient. Thus the use amount of the cyclodextrins may be determined in consideration of the weight or volume of the composition with respect to a unit dose of the compound and the like. However, such amount is preferably 0.5 to 50 parts by weight, more preferably 1 to 20 parts by weight, with respect to one part by weight of the cyclic peptide compound (I) or a salt thereof, though it varies depending upon the kind and the used amount of the cyclic peptide compound (I) or a salt thereof, its preparation form and/or the like.

As to the pharmaceutical composition of the present invention, the lyophilized composition is preferable.

The pharmaceutical composition of the present invention may be produced according to methods known in the art with using additives if necessary. Here, Basic Lecture on Development of Pharmaceuticals XI 20 Production of Pharmaceuticals (the second volume) (edited by Kyobunn Tsuda and Hisashi Nogami and published by Chizyo Shoten) is mentioned for reference.

The lyophilized composition may be obtained by preparing an aqueous solution of the cyclic peptide compound (I) ora salt thereof and cyclodextrins, optionally adding a pH buffer and/or a pH adjuster.

Suitable example of the pH buffer may be amino acid (e.g., glycine, L-arginine, L-aspartic acid, L-glutamic acid, etc.), sodium L-glutamate monohydrate, ethanolamine, trometamol, and the like, and the amount of the pH buffer is preferably 1 to 30 parts by weight, more preferably 10 to 20 parts by weight, with respect to one part by weight of the cyclic peptide compound (I) or a salt thereof, though it varies depending upon the kind and the used amount of the cyclic peptide compound (I) or a salt thereof, its preparation form and/or the like.

Suitable example of the pH adjuster may be inorganic base (e.g., sodium hydroxide, potassium hydroxide, calcium hydroxide, magnesium hydroxide), organic base (e.g., triethylamine, diisopropylethylamine, pyridine, picoline, ethanolamine, triethanolamine, dicyclohexylamine, N,N′-dibenzylethylenediamine, etc.), inorganic acid (e.g., hydrochloric acid, hydrobromic acid, sulfuric acid, phosphoric acid, etc.), organic carboxylic or sulfonic acid (e.g., formic acid, acetic acid, trifluoroacetic acid, maleic acid, tartaric acid, fumaric acid, methanesulfonic acid, benzenesulfonic acid, toluenesulfonic acid, etc), citric acid anhydrous, and the like, as required to attain pH 2.0 to 4.0, preferably pH 2.0 to 3.0, and then lyophilizing the resulting solution in vial according to a conventional method. Thus, the pharmaceutical composition in lyophilized form, when dissolved in purified water, preferably gives a solution of pH 2.0 to 4.0, more preferably pH 2.0 to 3.0.

It is preferable that the thus prepared composition in lyophilized form is sealed and stored with shading. The lyophilized composition can be loaded in each vial in the solution form before lyophilizing or in lyophilized powder form after lyophilizing.

Usually the pharmaceutical composition in lyophilized form is dissolved in isotonic sodium chloride solution as required and used as an injection solution. The pharmaceutical composition of the present invention maybe used as an injection preparation which requires some compounding before use.

The present invention is now described in further detail by way of Compositional Examples and Test Example by using the compounds 1 to 4 shown below, which should not be construed to limit the scope of the invention.

| | |
|---|---|
| The compound 2 | 55 mg |
| γ-Cyclodextrin | 70 mg |
| *Sodium citrate buffer solution | a suitable amount |

γ-cyclodextrin was dissolved in sodium citric buffer solution(5 mL). The compound 2 described below was dispersed into the solution, and stirred for about 2 hours. This suspension was centrifuged (relative centrifugal force: 1600 g, e. g., radius: 16 cm, speed: 3000 rpm) for 10 minutes, and the supernatant was taken. This solution contains about 10 mg/mL of the compound 2.

*: 2.1 g of citric acid monohydrate was dissolved in 1000 mL of water. The pH of this solution was adjusted to 4.2 with sodium hydroxide solution (1 mol/L or 0.1 mol/L).

### COMPOSITIONAL EXAMPLE 2

The composition containing 10 mg/mL of the compound 1 is obtained according to a similar manner to that of Compositional Example 1.

### COMPOSITIONAL EXAMPLE 3

The composition containing 10 mg/mL of the compound 3 is obtained according to a similar manner to that of Compositional Example 1.

### COMPOSITIONAL EXAMPLE 4

The composition containing 10 mg/mL of the compound 4 is obtained according to a similar manner to that of Compositional Example 1.

| | |
|---|---|
| The compound 2 | 55 mg |
| 2-hydroxypropyl-β-Cyclodextrin | 70 mg |
| *Sodium citrate buffer solution | a suitable amount |

2-hydroxypropyl-β-Cyclodextrin was dissolved in sodium citric buffer solution(5 mL). The compound 2 was dispersed into the solution, and stirred for about 2 hours. This suspension was centrifuged (relative centrifugal force: 1600 g, e. g., radius: 16 cm, speed: 3000 rpm) for 10 minutes, and the supernatant was taken. This solution contains about 10 mg/mL of the compound 2.

*: 2.1 g of citric acid monohydrate was dissolved in 1000 mL of water. The pH of this solution was adjusted to 4.2 with sodium hydroxide solution (1 mol/L or 0.1 mol/L).

### COMPOSITIONAL EXAMPLE 6

The composition containing 10 mg/ml of α-cyclodextrin is obtained according to a similar manner to that of Compositional Example 5

### COMPOSITIONAL EXAMPLE 7

The composition containing 10 mg/mL of β-cyclodextrin is obtained according to a similar manner to that of Compositional Example 5.

### COMPOSITIONAL EXAMPLE 8

The composition containing 10 mg/mL of sulfobutylether-β-cyclodextrin is obtained according to a similar manner to that of Compositional Example 5.

### COMPOSITIONAL EXAMPLE 9

The composition containing 10 mg/mL of δ-cyclodextrin is obtained according to a similar manner to that of Compositional Example 5.

### TEST EXAMPLE 1

The stability of the cyclic peptide compound (I)

0.5 mL of each solution of the compound 2 prepared by Compositional Examples 1, 5 and 8 was dissolved in physiological saline (to make 10 mL). 2 mL of this solution was each transferred into 3-mL glass vial. The resulting solution was each maintained at 25° C. under 1000 lux. After 24 hours after, the resulting solution was each tested on the residual amount of the compound 2. As a control, *the solution of the compound 2 solution without the cyclodextrins was used. The results are shown in Table 1.

*: 25 mg of the compound 2 was dissolved in 5 mL of glycine solution (50 mg/mL, pH9.8). Physiological saline was added to the solution to make 50 mL.

TABLE 1
Residual amount(%) of the compound 2

| Compositions | 0 hour | After 24 hours |
|---|---|---|
| Control | 100.0 | <75 |
| Compositional | 100.0 | >90 |
| Example 1 | | |
| Compositional | 100.0 | >90 |
| Example 5 | | |
| Compositional | 100.0 | >90 |
| Example 8 | | |

As is obvious from Table 1, the solutions of Compositional Examples 1, 5 and 8 were significantly stable as compared with the control solution.

The compounds 1 to 4 are shown below. They were obtained according to a similar manner to that of WO 01/60846.

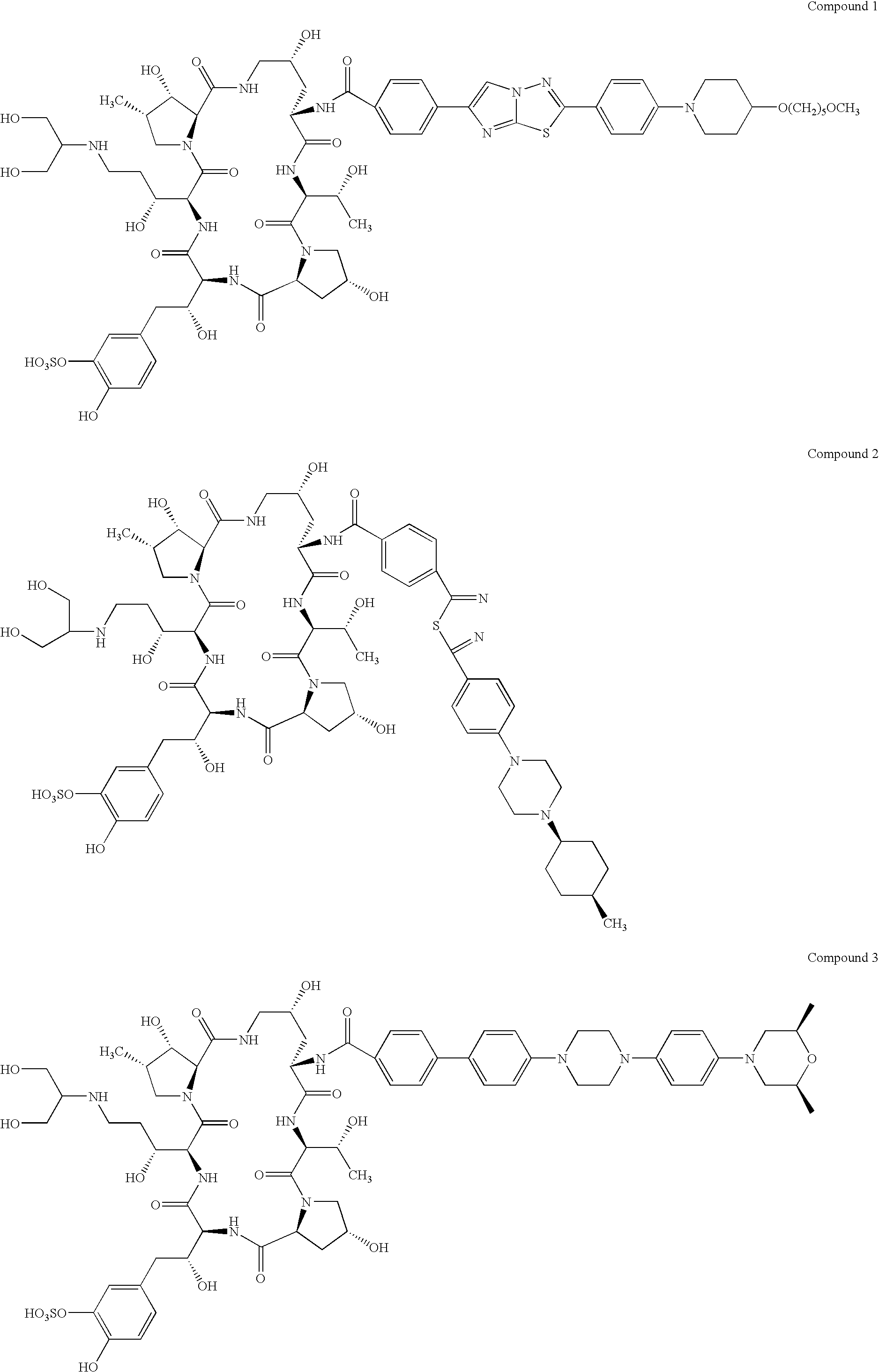

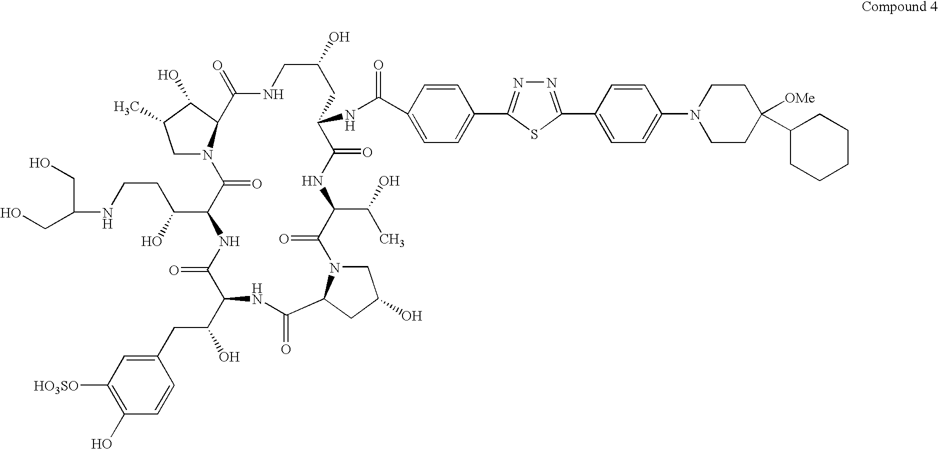

The cyclic peptide compound (I) or a salt thereof has an antifungal activity, particularly against the following-fungi.

Acremonium;

Absidia (e.g., Absidia corymbifera, etc);

Aspergillus (e.g., Aspergillus clavatus, Aspergillus flavus, Aspergillus fumigatus, Aspergillus nidulans, Aspergillus niger, Aspergillus terreus, Aspergillus versicolor, etc);

Blastomyces (e.g., Blastomyces dermatitidis, etc);

Candida (e.g., Candida albicans, Candida glabrata, Candida guilliermondii, Candida kefyr, Candida krusei, Candida parapsilosis, Candida stellatoides, Candida tropicalis, Candida utilis, etc.);

Cladosporium (e.g., Cladosporium trichoides, etc);

Coccidioides (e.g., Coccidioides immitis, etc);

Cryptococcus (e.g., Cryptococcus neoformans, etc);

Cunninghamella (e.g., Cunninghamella elegans, etc);

Dermatophyte;

Exophiala (e.g., Exophiala dermatitidis, Exophiala spinifera, etc);

Epidermophyton (e.g., Epidermophyton floccosum, etc);

Fonsecaea (e.g., Fonsecaea pedrosoi, etc);

Fusarium (e.g., Fusarium solani, etc);

Geotrichum (e.g., Geotrichum candiddum, etc);

Histoplasma (e.g., Histoplasma capsulatum var. capsulatum, etc);

Malassezia (e.g., Malassezia furfur, etc);

Microsporum (e.g., Microsporum canis, Microsporum gypseum, etc);

Mucor;

Paracoccidioides (e.g., Paracoccidioides brasiliensis, etc);

Penicillium (e.g., Penicillium marneffei, etc);

Phialophora;

Pneumocystis (e.g., Pneumocystis carinii, etc);

Pseudallescheria (e.g., Pseudallescheria boydii, etc);

Rhizopus (e.g., Rhizopus microsporus var. rhizopodiformis, Rhizopus oryzae, etc);

Saccharomyces (e.g., Saccharomyces cerevisiae, etc);

Scopulariopsis;

Sporothrix (e.g., Sporothrix schenchii, etc);

Trichophyton (e.g., Trichophyton mentagrophytes, Trichophyton rubrum, etc);

Trichosporon (e.g., Trichosporon asahii, Trichosporon cutaneum, etc).

The above fungi are well known to cause various infection diseases in skin, hair, nail, oral mucosa, gastrointestinal tract, bronchus, lung, endocardium, brain, meninges, urinary organ, vaginal protion, oral cavity, ophthalmus, systemic, kidney, bronchus, heart, external auditory canal, bone, nasal cavity, paranasal cavity, spleen, liver, hypodermal tissue, lymph duct, gastrointestine, articulation, muscle, tendon, interstitial plasma cell in lung, and so on.

Therefore, the present composition comprising the cyclic peptide compound (I) or a salt thereof is useful for preventing and treating various infectious diseases, such as dermatophytosis (e.g., trichophytosis, etc), pityriasis versicolor, candidiasis, cryptococcosis, geotrichosis, trichosporosis, aspergillosis, penicilliosis, fusariosis, zygomycosis, sporotrichosis, chromomycosis, coccidioidomycosis, histoplasmosis, blastomycosis, paracoccidioidomycosis, pseudallescheriosis, mycetoma, mycotic keratitis, otomycosis, pneumocystosis, and so on.

The present invention also provides a commercial package comprising the cyclic peptide compound (I) or a salt thereof of the present composition and a written matter associated therewith, wherein the written matter states that the pharmaceutical composition can or should be used for preventing or treating infections disease.

1. A pharmaceutical composition which comprises:
a cyclic peptide compound represented by the following general formula (I) 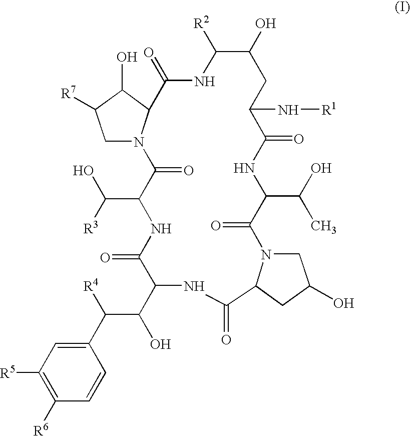
wherein
R¹ is hydrogen or acyl group,
R² is hydrogen, hydroxy or —NH(CH₂)₂NH₂,
R³ is methyl, —CH₂CONH₂, —(CH₂)₂NH₂ or
lower alkylamino(lower) alkyl substituted with one more hydroxy,
R⁴ is hydrogen or hydroxy,
R⁵ is hydrogen, hydroxy, lower alkoxy or hydroxysulfonyloxy,
R⁶ is hydroxy or acyloxy and,
R⁷ is hydrogen or methyl,
or a salt thereof as an active ingredient, and cyclodextrins.

2. A composition according to claim 1, which further comprises a pH adjuster and /or a pH buffer.

3. A composition according to claim 2, in which the cyclodextrins is α-cyclodextrins, β-cyclodextrins, γ-cyclodextrins or δ-cyclodextrins.

4. A composition according to claim 3, in which the cyclodextrins is α-cyclodextrin, β-cyclodextrin, hydroxypropyl-β-cyclodextrin, sulfobutylether-β-cyclodextrin, γ-cyclodextrin or δ-cyclodextrin.

5. A composition according to claim 2, in which the pH buffer is amino acid.

6. A composition according to claim 5, in which the amino acid is L-arginine.

7. A composition according to claim 1, which contains 0.5 to 50 parts by weight of the cyclodextrins with respect to one part by weight of the cyclic peptide compound (I) or a salt thereof.

8. A composition according to claim 1, which contains 0.1 to 400 mg of the cyclic peptide compound (I) or a salt thereof in a single unit dose.

9. A composition according to claim 1 prepared by the steps of:
dissolving the cyclic peptide compound (I) or a salt thereof, the cyclodextrins and optionally a pH adjuster and/or a pH buffer in a purified water, and lyophilizing the solution.

10. A composition of claim 1, which, when dissolved in purified water, gives a solution of pH 2.0 to 4.0.

11. An injection preparation prepared by dissolving the composition of claim 1 in isotonic sodium chloride solution.

12. A commercial package comprising the pharmaceutical composition of any one of claim 1 to claim 11 and a written matter associated therewith, wherein the written matter states that the pharmaceutical composition can or should be used for preventing or treating infections disease.
